(19) United States
(12) Patent Application Publication
(10) Pub. No.: US 2021/0205572 A1
(43) Pub. Date: Jul. 8, 2021

(54) THERAPEUTIC USES OF DIGITAL STORY CAPTURE SYSTEMS

(71) Applicant: Ionic Ventures, LLC, San Francisco, CA (US)

(72) Inventors: David KEENE, San Diego, CA (US); Yevgeniy KOSTIKOV, San Diego, CA (US); Edward COX, San Diego, CA (US)

(21) Appl. No.: 16/905,556

(22) Filed: Jun. 18, 2020

Related U.S. Application Data
(63) Continuation of application No. 15/850,386, filed on Dec. 21, 2017, now abandoned.
(60) Provisional application No. 62/438,445, filed on Dec. 22, 2016.

(51) Int. Cl. A61M 21/00; A61B 5/16; A61B 5/00

(52) U.S. Cl. CPC A61M 21/00; A61B 5/165; A61B 5/486; A61B 5/11; A61M 2021/005; A61M 2021/0027; A61M 2230/18; A61B 5/163

(57) ABSTRACT

Disclosed herein are methods of reducing anxiety and/or agitation in a subject, with a neurological disorder, or a subject undergoing therapy, comprising exposing the subject to reminiscence therapy via a digital therapeutic device. The digital therapeutic device can include a transceiver configured to communicate with a database and a first user device and a processor operatively coupled to the user interface, a microphone, a speaker, and the transceiver.

### CROSS-REFERENCE TO RELATED APPLICATIONS

This application is a continuation of U.S. application Ser. No. 15/850,386, filed Dec. 21, 2017, which claims the benefit of the U.S. Provisional Patent Application No. 62/438,445 filed Dec. 22, 2016, the contents of which are hereby incorporated by reference herein in their entireties.

### FIELD

This disclosure is in the field of methods and devices for the treatment of neurological disorders in human subjects, particularly those disorders that originate in the brain.

### BACKGROUND

The following description is provided to assist the understanding of the reader. None of the information provided or references cited is admitted to be prior art. Neurological disorders can be caused by genetic disorders, congenital abnormalities or disorders, infections, lifestyle or environmental health problems including malnutrition, brain injury, spinal cord injury and/or nerve injury. Neurodegenerative disorders are hereditary and/or idiopathic conditions characterized by progressive nervous system dysfunction that result in progressive degeneration and/or death of nerve cells. Their etiology is not yet fully understood. The current state of the art in treating neurological disorders involves either drugs or the open-loop electrical stimulation of neurologic tissue. Drug therapy has been shown to have significant short and long term side effects and is often ineffective. As such, new non-drug treatments are needed.

### SUMMARY

Disclosed herein, in some embodiments, is a method of reducing symptoms of anxiety and/or agitation in a subject, including, but not limited to those with a neurological disorder. The methods can include, for example, exposing the subject to the content of a digital therapeutic, wherein the digital therapeutic comprises digital content comprising photographs, sound, and/or video, and wherein the digital content is thematically related to the subject's life. In some embodiments, the neurological disorder is selected from Alzheimer's disease, Dementia, Post-Traumatic Stress Disorder (PTSD), Schizophrenia, Parkinson's disease, general depression, and/or general anxiety.

Disclosed herein, in some embodiments, is a method of slowing the progression of a neurological disorder in a subject, comprising exposing the subject to the content of a digital therapeutic, wherein the digital therapeutic comprises digital content, wherein the digital content comprises photographs, sound, and/or video, and wherein the digital content is thematically related to the subject's life.

In some embodiments, the neurological disorder is selected from Alzheimer's disease, Dementia, Post-Traumatic Stress Disorder (PTSD), Schizophrenia, Parkinson's disease, general depression, and/or general anxiety.

Disclosed herein, in other embodiments, is a method of reducing symptoms of anxiety and/or agitation in a subject undergoing a therapy, comprising exposing the subject to the content of a digital therapeutic, wherein the digital therapeutic comprises digital content, wherein the digital content comprises photographs, sound, and/or video, and wherein the digital content is thematically related to the subject's life, wherein the therapy is selected from drug rehab therapy, suicide prevention, dignity therapy (e.g., for cancer patients undergoing chemotherapy), occupational therapy, couples counseling, or other long term care that requires separation from the family.

In some embodiments, the subject is exposed to the content of a digital therapeutic on a regular basis.

In some embodiments, the quality of life (QOL) of the subject is improved following exposure to a digital therapeutic.

In some embodiments, the methods disclosed herein further comprise monitoring a patient's reaction to a digital therapeutic, wherein monitoring a patient's reaction comprises detection of movement of the patient's facial features and/or eye movements. In some embodiments, the methods further comprise optimizing the content of the digital therapeutic device to enhance positive facial movements and/or to increase eye gaze to the device.

In some embodiments, the method of optimizing content in a digital therapeutic for a patient with a neurological disorder comprises monitoring the patient while the patient is exposed to a digital therapeutic, wherein monitoring the patient comprises detection of movement of facial features and/or eye movements, and increasing amount of content in the digital therapeutic that results in favorable movement of facial features and/or increased gaze upon the digital therapeutic.

In some embodiments, an illustrative digital therapeutic device for use in the disclosed methods includes a user interface configured to display information and receive user input, a microphone configured to detect sound, and a speaker configured to transmit sound. The device can also include a transceiver configured to communicate with a database and a first user device and a processor operatively coupled to the user interface, the microphone, the speaker, and the transceiver. The processor is configured to receive a first image from the database and receive from the first user device a first message. The first message includes a request for information related to the first image. The processor is also configured to record via the microphone an audio recording that includes information related to the first image, transmit the audio recording to the database, and transmit to the database a request for the first image. The processor is further configured to receive the first image with an identifier of the audio recording and cause the user interface to display the first image and simultaneously cause the speaker to play the audio recording.

A digital therapeutic device comprising: a user interface configured to display information and receive user input; a microphone configured to detect sound; a speaker configured to transmit sound; a video camera configured to record a patient's reaction to digital therapeutic content presented on the user interface; a transceiver configured to communicate with a database and a first user device; and a processor operatively coupled to the user interface, the microphone, the speaker, and the transceiver, wherein the processor is configured to: receive a first image from the database; receive from the first user device a first message, wherein the first message includes a request for information related to the first image; record via the microphone an audio recording that includes information related to the first image; transmit the audio recording to the database; transmit to the database a request for the first image; receive the first image with an identifier of the audio recording; and cause the user interface to display the first image and simultaneously cause the speaker to play the audio recording. In some embodiments, the processor is further configured to: cause the user interface to simultaneously display the first image and a plurality of messages, wherein the plurality of messages includes the first message; and receive from the user interface an indication that the first message was selected. In some embodiments, the processor is further configured to receive from the user interface an indication that the first message is to be sent to the first user device. In some embodiments, to receive the first image with the identifier of the audio recording, the processor is configured to receive the first image with the identifier of the audio recording and a second message, wherein the second message comprises text information related to the first image, and wherein the processor is configured to cause the user interface to simultaneously display the first image and the second message.

In some embodiments, the device further comprises an first image capture device, and the processor is further configured to: receive the first image from the first image capture device; and transmit the first image to the database. In some embodiments, to transmit the audio recording to the database, the processor is configured to parse the audio recording into a plurality of audio files and transmit the plurality of audio files to the database individually. In some embodiments, the first image is one of a plurality of images that comprises a video. In some embodiments, the processor is further configured to: receive from a second user device a third message that comprises a request for information related to a second image; and cause the user interface to simultaneously display the second image and the third message. In some embodiments, the processor is further configured to: receive from the second user device the audio recording, wherein the audio recording includes information related to the image and was recorded by the second user device; cause the memory to store the audio recording with an indication that relates the audio recording to the image; receive from the first user device a request for the image; and in response to receiving the request for the image, transmit to the first user device the image and an identifier of the audio recording. In some embodiments, the processor is further configured to receive from the first user device an indication that the first message is to be sent to the second user device. In some embodiments, the processor is further configured to receive from a third user device a second message that comprises text information related to the image, and wherein to transmit the image and the identifier, the processor is configured to transmit the image, the identifier, and the second message. In some embodiments, the processor is further configured to: receive from the first user device the first image, wherein the first image was captured by the first user device; and transmit to the database the first image. In some embodiments, the processor is further configured to detect movement of the patient's facial features and/or eye movements. In some embodiments, the processor is further configured to optimize the content of the user interface to enhance positive facial movements and/or to increase eye gaze to the device. In some embodiments, the processor is configured to increase content in the digital therapeutic that results in favorable movement of facial features and/or increased gaze upon the digital therapeutic.

Another aspect provided herein is a method comprising: receiving a first image from a database; receiving from a first user device a first message, wherein the first message includes a request for information related to the first image; recording via a microphone an audio recording that includes information related to the first image; recording via a video camera a patient's reaction to digital therapeutic content presented on a user interface; transmitting the audio recording to the database; transmitting to the database a request for the first image; receiving the first image with an identifier of the audio recording; and causing a user interface to display the first image and simultaneously cause a speaker to play the audio recording. In some embodiments, the method further comprises receiving from a second user device a third message that comprises a request for information related to a second image; and causing the user interface to simultaneously display the second image and the third message. In some embodiments, the method further comprises receiving from the second user device the audio recording, wherein the audio recording includes information related to the image and was recorded by the second user device; causing the memory to store the audio recording with an indication that relates the audio recording to the image; receiving from the first user device a request for the image; and in response to receiving the request for the image, transmitting to the first user device the image and an identifier of the audio recording. In some embodiments, the method further comprises detecting movement of facial features of the patient. In some embodiments, the method further comprises optimizing content to the user interface to enhance positive facial movements and/or increase eye gaze to the user interface.

The foregoing summary is illustrative only and is not intended to be in any way limiting. In addition to the illustrative aspects, embodiments, and features described above, further aspects, embodiments, and features will become apparent by reference to the following drawings and the detailed description.

### DETAILED DESCRIPTION

Disclosed herein are methods of using a digital therapeutic device for reminiscence therapy in certain subjects and/or patients.

Reminiscence may be used therapeutically to treat physical or emotional illness or injury. By soliciting explicit and implicit memories, therapeutic reminiscence may provide a structured and enjoyable activity; promote intrapersonal and interpersonal functioning; stimulate cognition, memories and emotions; and improve indicators of well-being. For example, therapeutic reminiscence may be beneficial for people undergoing physical, cardiac or cognitive rehabilitation. Reminiscence may also be used to improve well-being while minimizing illness or injury. For example, therapeutic reminiscence may be used to strengthen military families for the physical, psychological and emotional rigors of deployment far from home.

While most branches of health care focus on prevention and treatment of disease, rehabilitation principally focuses on the enhancement of human functioning and quality of life. In a broad sense, rehabilitation attempts to tackle the complex relationship between disease and the ability to function: eradicating disease does not necessarily eliminate disability; likewise, disability can be minimized even in the face of permanent injury or chronic disease.

For example, memories useful in reminiscence therapy can include a childhood home, love interests, schooling, summer camps, friends and family, etc. Reminiscence therapy, including showing the adult image reminders of these memories, allows an adult to revisit those memories to reinforce self-identity.

Disclosed herein, in some embodiments, is a method of decreasing anxiety or agitation in a subject or patient comprising administration of reminiscence therapy to the subject or patient via a digital therapeutic device. In addition, disclosed herein, are methods for increasing ‘non-patient user’, or content provider participation and methods for measuring the effect of a digital therapeutic on the subject or patient via data monitoring and analysis.

In some embodiments, the reminiscence therapy is delivered via a digital player device to patient. In some embodiments, a ‘non-patient user’ or content provider interacts with the system via a digital therapeutic device to tell audio stories, provide photograph and video content.

User and system provided content for reminiscence therapy includes but is not limited to pictures, art, spoken audio, music audio, therapeutic sounds and videos.

In some embodiments, the content provided by the content providers is tagged with the user who provided the content, the voice that is heard on a recording, and/or the theme of the content. For example, a script can be used as a starting point of the recoding (in audio).

Additionally, in some embodiments, the digital therapeutic device asks a content provider to provide additional metadata about content at various times during the device's use. Examples of this additional metadata include, but are not limited to, faces present in the content, face location in the content, date of capture, date of captured event, how the content makes the user feel, and/or relationships of subjects in photos.

Additionally, in some embodiments, the digital therapeutic device will automatically detect features in the photos and videos that do not require user interaction. Examples of these features include, but are not limited to, the presence and location of landmarks, the presence and location of animals, the grain and color aspects of the photograph, the steadiness of the camera, the sharpness of focus, the presence and location of recognized faces, time of eye gaze on various elements in the content, location of pausing in video, common or featured colors in content, and/or angular and organic shapes in the content.

Additionally, in some embodiments, the digital therapeutic device will monitor the patient and record images and videos of the patient while watching the content. In some embodiments, the image capture will be taken when the device recognizes various emotions and detects interaction, such as speech.

### Neurological Disorders

In some embodiments, disclosed herein is a method of treating a subject, or patient, with a neurological disorder by exposing the subject or patient to a digital therapeutic, as disclosed herein. In some embodiments, the neurological disorder is a neurodegenerative condition.

As used herein, the term “neurodegenerative condition” refers to a degeneration of neurons in either the brain or the nervous system of an individual. Non-limiting examples include Amyotrophic lateral sclerosis (ALS), Alzheimer's disease (AD), Parkinson's Diseases (PD), multiple sclerosis (MS), dementia, and frontotemporal dementia. Neurodegenerative conditions also include traumatic brain injury (TBI), mild brain injury (mBI), recurrent TBI, and recurrent mBI. In some embodiments, the neurodegenerative condition is caused by a reperfusion injury. In some embodiments, the reperfusion injury is resulted by post cardiac arrest, coronary artery bypass grafting (CABG), ischemia, anoxia, or hypoxia. Neurodegenerative conditions are debilitating, the damage that they cause can be irreversible, and the outcome in a number of cases is fatal.

In some embodiments, the methods disclosed herein when applied to a subject with a neurodegenerative disorder, result in an increased quality of life (QOL) for the subject.

### Subjects in Various Therapies

In some embodiments disclosed herein is a method of reducing anxiety or agitation in a subject or patient undergoing therapy, comprising exposing the subject or patient to a digital therapeutic device, as disclosed herein. In some embodiments, the therapy is cognitive or psychotherapy. In some embodiments, the therapy is selected from drug rehab therapy, suicide prevention, dignity therapy (e.g., for cancer patients undergoing chemotherapy), occupational therapy, couples counseling, or other long term care that requires separation from the family.

In some embodiments, the methods disclosed herein when applied to a subject with a neurodegenerative disorder, result in an increased quality of life (QOL) for the subject. In some embodiments, the methods disclosed herein result in greater adherence to said therapy.

### Optimization of Digital Therapeutic Content

In a preferred embodiment, a method of optimizing content of a digital therapeutic device involves monitoring play statistics on the player device that include but are not limited to play time, repeat count, skip button iteration and approval buttons and ratings. These play statistics are used in conjunction with metadata to calculate aspects of the content which provide the greatest therapeutic effect to the patient. These data are also used to discover the patient's interest in aspects of content.

In some embodiments, the method of optimizing content of a digital therapeutic device involves using the device's cameras to monitor the patient reaction and attention to content. Examples of this monitoring include, but are not limited to: face detection, emotion detection such as smiles or surprise, eye tracking, gaze or eye linger tracking. These data can be used in conjunction with metadata, including location of features to calculate aspects of the content which provide the greatest therapeutic effect to the patient. These data are also used to discover the patient's interest in aspects of content.

In some embodiments, the method of optimizing content of a digital therapeutic device involves using device telemetry to monitor patient reaction and attention. Examples of this monitoring include, but are not limited to, device angle, hand steadiness, sudden movement and screen taps. These data are used in conjunction with metadata, including location of features to calculate aspects of the content which provide the greatest therapeutic effect to the patient. These data are also used to discover the patient's interest in aspects of content.

In some embodiments, the method of optimizing content of a digital therapeutic device involves using explicit questions to monitor patient reaction and attention. Examples of this monitoring include, but are not limited to, asking the patient questions with one word answers that require a single click, asking the patient to answer simple questions by talking to the device. These data can be used in conjunction with all of the metadata listed above, including location of features to calculate aspects of the content which provide the greatest therapeutic effect to the patient. These data also can be used to discover the patient's interest in aspects of content.

Patient enjoyment, relaxation and interest in content can be stored and processed algorithmically to produce feature/enjoyment vectors for all provided content. Using the data, therapeutic videos are re-edited by the system and sent to the player device. Over time, increased or decreased attention and therapeutic effect will be tracked and content will be adjusted accordingly.

### Increasing Non-Patient Content Provider Participation

In some embodiments, disclosed herein is a method of increasing non-patient content provider participation in providing content of a digital therapeutic device, when the digital therapeutic device is used for a therapeutic method.

In some embodiments, the method involves using an engaging chat via artificial intelligence (AI) to mimic the role of a human (e.g., therapist) to ask the content provider for content. Interspersed with content requests are occasional rewards and encouragement. Encouragement includes, but is not limited to, playing back reaction photos and videos of the patient's enjoyment, educating the users about the therapeutic effect their work is having, and providing statistics of how much content the user and the user's peers in the family group have provided.

In some embodiments, the method involves analyzing at what times user interaction with an application is the highest, and prioritizing communication with the user at that time.

In some embodiments, the method involves calculating via an algorithm which content the user is most interested in and prioritizing questions about that content. Interest is calculated by, but not limited to, number and length of audio recorded in the past on content with similar features, number of views of content with features, content uploaded by the user with similar features.

### Digital Therapeutic Devices and Uses thereof

In some embodiments, the therapeutic methods disclosed herein comprise use of a digital therapeutic device for administration of reminiscence therapy. In some embodiments, the digital therapeutic device is a computerized story capture system.

In some embodiments, a computerized story capture system provides a digital service that makes it easy to create a high fidelity digital archive of a family's stories for preservation for the next generation. In some embodiments, the computerized story capture system allows people to browse through photos while recording audio of the stories as they are organically told. In some embodiments, the computerized story capture system permits the user to naturally tell the story by choosing any photos they wish instead of only being able to record the audio over photos in a pre-ordered way such as a slideshow. A detailed description of exemplary embodiments of story capture system devices, and uses thereof, is disclosed in U.S. Patent Publication No. 2016/0267081, which is hereby incorporated by reference herein in its entirety.

In some embodiments, the computerized story capture system enables users to record long running audio with no time limits and link that audio to photos to add context to the stories being told. Users can playback this audio as recorded (linear playback) or mixed with audio recorded at a different date (non-linear playback).

By way of example, a user could listen to all the audio recorded while the people speaking were looking at a particular image. The playback for a particular photo would play audio from 1:12:00 of a first two-hour recording session, 0:45:00 of a second one-hour recording session, and 00:01:00 of a third three-hour session. In a preferred embodiment, the audio is stored in a networked storage system, such as “the cloud,” not locally to the playback device.

Some embodiments of a computerized story capture system provide several advantageous features. For example, a preferred embodiment allows a user to quickly download and seek to a specific point in each audio session without incurring the latency and bandwidth costs of downloading the whole clip. Some embodiments avoid holding open communication connections for streaming during recording and playback.

In an illustrative embodiment, user devices such as smartphones can be used to send an image to other user devices with a request for information regarding the image. For example, Amy can send an image of her grandfather to Steve, Amy's uncle. The image can be of Amy's grandfather holding a large fish in front of a lake. Steve can receive the image on his smartphone with a request from Amy asking Steve to explain the context of the image. In an illustrative embodiment, Steve can provide a response to Amy in the form of text, such as, “This photo was taken on one of our annual fishing trips to Canada when I was a kid.” In an alternative embodiment, Steve can record, via his smartphone, himself telling a story about the photo. For example, Steve can discuss the trip to Canada, how his dad struggled to get the fish into the boat, and how Steve was so excited that his hands were shaking when he took the photo of his dad, which explains why the photo is blurry. The explanation of the photo (e.g., whether in text format or audio format) can be stored in connection with the image. In an illustrative embodiment, Amy, her sisters, and other family members can access the photo and the explanation at a later time to reminisce, thereby preserving the memory.

As explained in greater detail below, various embodiments described herein provide functions and features that were not previously possible. For example, in some embodiments, a slideshow or photo album is presented to a user that includes a narration of one or more photos. The content of the slideshow or photo album can be accessed electronically virtually anywhere and at any time regardless of the availability of the narrator (e.g., whether the narrator is busy, ill, or deceased).

In some embodiments, a slideshow or photo album with associated audio recordings can provide advantages that were not previously available. For example, audio recordings can allow a person to explain the context and story surrounding a photo that would not be known by simply viewing the photo. Also, prompting a narrator for details about a photo or a story can allow the narrator to remember additional details, stories, or context that the narrator would not have otherwise provided. Recording such content preserves the stories and context in a manner that captures more of the emotion regarding the photo, story, or narrator than a simple photo or text-based explanation can. Additionally, various embodiments described herein make it more convenient and easier for people to record their stories or explanations of photos, thereby increasing, for example, the amount of familial history that is preserved. For example, very few individuals write memoirs about their life for their family members to cherish because it can be difficult or the individuals are uninterested in writing a memoir. However, various embodiments described herein make it easy for anyone to record stories and their own history. Furthermore, many people enjoy telling stories but do not enjoy writing.

Thus, various embodiments can be used to capture and preserve memories by making replay of the memories more enjoyable. Many people find it easier and more compatible with the human sensory system to watch and listen (e.g., to a slideshow of family histories while listening to a family member describe the photos) than to read a memoir. For example, it can be more enjoyable to listen to a story with a slideshow of relevant pictures than to sit and read a memoir. Various embodiments can make it easier to record their memories by simply telling a story related to associated photos.

In some embodiments, image and audio data is stored on one or more servers and transmitted to a user device in segments, thereby reducing the amount of information transmitted to and stored on the user device. In an illustrative embodiment, audio recordings are associated with one or more images. Similarly, in such embodiments, an image can be associated with one or more audio recordings or portions of audio recordings. A database or record can be kept (e.g., on a server of the network, on the image storage device, on the audio storage device, etc.) that maintains such associations between images and audio recordings (or segments of audio recordings). In response to a user device requesting to download an image, a server of the network can check such a database or record to determine associated audio recordings. The server can transmit to the user device the image and a listing of the associated audio recordings. Similarly, in response to a user requesting to play an audio recording, the server can transmit to the user device a listing of the images associated with the audio recording.

In an illustrative embodiment, metadata associated with the audio recording can include an indication of who is speaking. For example, an audio recording can include multiple people speaking about a photo. The metadata can be used to indicate who is speaking at any particular instance. A user can add or edit the metadata to include names of individuals and when individuals begin and/or stop speaking. In an illustrative embodiment, during the recording, a user can select one of a plurality of individuals to indicate who is speaking. The selection of the individuals can be stored as metadata of the audio recording. During replay of the audio recording, an indication of who is speaking (e.g., who was selected during the recording) can be displayed.

In an illustrative embodiment, metadata associated with screen touches can be stored with the audio recording. For example, while recording, the user device tracks where a user taps or gestures on the photo during the recording. The user device records the places where the user has tapped or interacted with a displayed image. During playback, the touches or interactions with the touch screen can be displayed. In some embodiments, recognized gestures such as shapes cause a function to be performed, such as displaying a graphic. Interactions with the image can include zooming in or out, circling faces, drawing lines, etc.

In an illustrative embodiment, along with the audio recording, the user device can record a video of the user during the audio recording. The video can be played back during the playback of the audio recording. For example, a viewing window can be displayed for the video during playback while the image about which the subject is talking is simultaneously displayed. In an illustrative embodiment, the viewing window is displayed on the screen while the audio and video are recording. The user can move the viewing window around the screen during recording (e.g., to view a portion of the image that is obstructed by the viewing window). The location of the viewing window during the audio recording can be recorded and played back during the audio playback. Thus, the viewer of the playback can see the same screen that was displayed during the recording.

In an illustrative embodiment, the user device can detect that during a recording, speaking has stopped. After a predetermined threshold of not detecting speech (e.g., ten seconds, twenty seconds, thirty seconds, one minute, ten minutes, etc.), the application can prompt the user to end the recording session (or continue the session). In an alternative embodiment, after a predetermined threshold of not detecting speech, a suggested question can be displayed to the user to facilitate explanation or storytelling. For example, a selected image during a recording session can be tagged with Grandpa and Aunt JoAnn. After a predetermined threshold of silence, a pop-up display can ask, “What was Grandpa doing in this picture?” or “How old was Aunt JoAnn in this picture?” The questions can be selected based on the tags of an image, dates of when the image was captured, etc.

In an illustrative embodiment, a user device records the audio files and the metadata and breaks the session into the multiple audio files (and associated metadata) into portions. The user device can upload the portions separately, thereby minimizing loss in the event of a communications malfunction or a computing crash. Uploading the portions separately minimizes the time that a streaming communication link is maintained, thereby increasing reliability of the communication.

In an illustrative embodiment, a user interface display is provided on a user device to allow the user to navigate audio stories without leaving the context of the photos themselves. For example, the computerized story capture system includes a playback screen that puts linear progression horizontally on the page and uses vertical space to represent other stories that are available within the current context.

In an illustrative embodiment, the various content (e.g., images, videos, audio recordings) can be organized in multiple ways to allow a user to navigate through the content. For example, the content can be found by selecting the person who uploaded the image or an album that the content is associated with.

In an illustrative embodiment, the various images in an album can be displayed using keywords that a user associates with images, locations of where the images were taken, people tagged in the images, dates of when the images were taken, etc. For example, images can be organized based on date ranges, such as decades (e.g., 1960s, 1970s, 1980s, etc.). In an alternative embodiment, the various images are organized by a popularity rating (e.g., based upon the number of times each image is viewed or downloaded). In an illustrative embodiment, images that have associated recordings can be marked as such. For example, a speech bubble can be displayed in the corner of the thumbnail of an image in an album.

In an illustrative embodiment, a user can request that another user input an annotation to an image. The annotation can be in the form of a short text answer, a long text answer, an audio recording, a video recording, etc. The annotation can be stored along with the image to be recalled later by either user or another user. In alternative embodiments, any suitable user device can be used such as a computer, a tablet, etc.

In an illustrative embodiment, a user can select a photo and transmit the photo to another user's user device for comment and/or annotation. For example, the user can ask a question related to the photo. For example, the user of the user device selected a photo that is displayed at the top of the user interface. On the bottom of the user interface, the user is presented with suggested questions. In an illustrative embodiment, the suggested questions are predetermined. In an alternative embodiment, at least some of the suggested questions are questions that the user previously asked another user. For example, the user can be asked to “Type a question” in an input box. The user can also be presented with questions that the user previously typed for another photo or another user. The suggested questions can include, for example, “What is happening here?”; “How did this make you feel?”; “Does this moment make you feel proud?”; and “If you could go back in time and tell yourself something on that day what would it be?” In an illustrative embodiment, the user can be presented with a button “Suggest new questions” that will re-populate the suggested questions with other suggested questions.

In an illustrative embodiment, multiple users can contribute to the creation of a story.

An illustrative embodiment can be used to capture stories by non-associated people, such as non-family members, nurses, staff, etc. For example, a woman in a nursing home can have one or more conditions that affect the woman's memory. However, the woman may have lucid moments in which she can remember events from her past. In an illustrative embodiment, a nurse or staff member of the nursing home can use an embodiment of the present disclosure to record a story told by the woman (e.g., during a lucid moment). In an illustrative embodiment, the nurse or staff member can use a user device such as a smartphone with an application installed that records the woman's story. In such an embodiment, the application can allow the nurse or staff member to record a story, but not allow the nurse or staff member to replay, delete, and/or edit the recording. For example, in some instances, family members may wish to have control over the recordings, not the nurse or staff member.

One or more of the embodiments described herein can contain an administrator mode that allows users such as nurses to record and store content to multiple accounts. For example, a nurse may be responsible for twenty patients. The nurse may have access to accounts associated with each of the twenty patients. The access of the nurse can be limited based on the preferences of each patient (or their family member). For example, the nurse may have the ability to record content and store the content, but not have the ability to delete content.

Such embodiments can be used in any suitable context. For example, a parent can have recorded a story such that another caretaker (e.g., a nurse while the child is in the hospital, a staff member of a daycare, another parent while the child is at a sleep-over, etc.) can replay the story and calm the child down (e.g., if the child is homesick or is missing his or her parents). In other examples, the replaying of stories can be used in any other therapeutic or clinical purposes. In such an embodiment, the nursing or staff members may have access to replay or view content, but may not have access to add or delete content. In alternative embodiments, the nurse or staff member can have any suitable amount or degree of control or privileges over the account.

In an illustrative embodiment, the digital therapeutic device comprises a process. The processor executes instructions. The instructions may be carried out by a special purpose computer, logic circuits, or hardware circuits. The processor may be implemented in hardware, firmware, software, or any combination thereof. The term “execution” is, for example, the process of running an application or the carrying out of the operation called for by an instruction. The instructions may be written using one or more programming language, scripting language, assembly language, etc. The processor executes an instruction, meaning that it performs the operations called for by that instruction. The processor operably couples with the user interface, the transceiver, the memory, etc. to receive, to send, and to process information and to control the operations of the computing device. The processor may retrieve a set of instructions from a permanent memory device such as a ROM device and copy the instructions in an executable form to a temporary memory device that is generally some form of RAM. An illustrative computing device may include a plurality of processors that use the same or a different processing technology. In an illustrative embodiment, the instructions may be stored in memory.

In an illustrative embodiment, the digital therapeutic device comprises a transceiver. The transceiver is configured to receive and/or transmit information. In some embodiments, the transceiver communicates information via a wired connection, such as an Ethernet connection, one or more twisted pair wires, coaxial cables, fiber optic cables, etc. In some embodiments, the transceiver communicates information via a wireless connection using microwaves, infrared waves, radio waves, spread spectrum technologies, satellites, etc. The transceiver can be configured to communicate with another device using cellular networks, local area networks, wide area networks, the Internet, etc. In some embodiments, one or more of the elements of the computing device communicate via wired or wireless communications. In some embodiments, the transceiver provides an interface for presenting information from the computing device to external systems, users, or memory. For example, the transceiver may include an interface to a display, a printer, a speaker, etc. In an illustrative embodiment, the transceiver may also include alarm/indicator lights, a network interface, a disk drive, a computer memory device, etc. In an illustrative embodiment, the transceiver can receive information from external systems, users, memory, etc.

In an illustrative embodiment, the user interface is configured to receive and/or provide information from/to a user. The user interface can be any suitable user interface. The user interface can be an interface for receiving user input and/or machine instructions for entry into the computing device. The user interface may use various input technologies including, but not limited to, a keyboard, a stylus and/or touch screen, a mouse, a track ball, a keypad, a microphone, voice recognition, motion recognition, disk drives, remote controllers, input ports, one or more buttons, dials, joysticks, etc. to allow an external source, such as a user, to enter information into the computing device. The user interface can be used to navigate menus, adjust options, adjust settings, adjust display, etc.

The user interface can be configured to provide an interface for presenting information from the computing device to external systems, users, memory, etc. For example, the user interface can include an interface for a display, a printer, a speaker, alarm/indicator lights, a network interface, a disk drive, a computer memory device, etc. The user interface can include a color display, a cathode-ray tube (CRT), a liquid crystal display (LCD), a plasma display, an organic light-emitting diode (OLED) display, etc.

In an illustrative embodiment, the power source is configured to provide electrical power to one or more elements of the computing device. In some embodiments, the power source includes an alternating power source, such as available line voltage (e.g., 120 Volts alternating current at 60 Hertz in the United States). The power source can include one or more transformers, rectifiers, etc., to convert electrical power into power useable by the one or more elements of the computing device, such as 1.5 Volts, 8 Volts, 12 Volts, 24 Volts, etc. The power source can include one or more batteries.

In an illustrative embodiment, the computing device includes a sensor. Said sensor can include an image capture device. In some embodiments, the sensor can capture two-dimensional images. In other embodiments, the sensor can capture three-dimensional images. The sensor can be a still-image camera, a video camera, etc. The sensor can be configured to capture color images, black-and-white images, filtered images (e.g., a sepia filter, a color filter, a blurring filter, etc.), images captured through one or more lenses (e.g., a magnification lens, a wide angle lens, etc.), etc. In some embodiments, sensor (and/or processor) can modify one or more image settings or features, such as color, contrast, brightness, white scale, saturation, sharpness, etc. In another example, the sensor is a device attachable to a smartphone, tablet, etc. In yet another example, the sensor is a device integrated into a smartphone, tablet, etc. In an illustrative embodiment, the sensor can include a microphone. The microphone can be used to record audio, such as one or more people speaking.

In an illustrative embodiment, any of the operations described herein can be implemented at least in part as computer-readable instructions stored on a computer-readable memory. Upon execution of the computer-readable instructions by a processor, the computer-readable instructions can cause a node to perform the operations.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present.

### EXAMPLES

### Example 1: Use of the Application as a Behavioral Intervention for Mild to Moderate Dementia

This is a Proof of Concept Study that will examine the feasibility of using a digital therapeutic, in the treatment of mood and cognitive symptoms associated with mild to moderate dementia. Individuals with dementia and their caregivers will be provided with a computer tablet and software that will allow them to view pictures of themselves and family members with narratives that are put to music. The primary goal of this project is to study the feasibility of introducing the tablet and software to the patient, caregiver, and at least two other family members (who will provide additional photos and audio) to determine whether the study participants are willing to provide content (in the form of pictures and audio) and use the tablet and software at home, and if so, determine how often they are willing to use the device and system during a 4-week period. In addition, our secondary goal is to examine changes in mood, quality of life, and cognition before and after the 4-week period in patients, as well as examine changes in mood, quality of life, and caregiver burden, in the patients' caregivers over the same period. The major concepts of this project are based on Reminiscence Therapy (RT), Cognitive-Reminiscence Therapy (CRT), and Life Review Therapy (LRT), which have shown to be effective in the treatment of cognitive and mood symptoms in patients with Alzheimer's disease (AD) and other forms of dementia.

This Proof of Concept study will examine the potential utility of a digital therapeutic, in the form of a computer tablet that contains pictures and narratives set to music as well as audio recordings from the patient's family and loved ones. The application is an online-based story-sharing platform that allows users to record audio over photos as a way to privately share and preserve their family history and personal legacy. The technology is unique in that it allows multiple family members, even if they are separated by time and place, to collaborate on the stories in just a few minutes a day. Participants will be 30 patients with mild to moderate dementia, their caregivers (30), and approximately 2-3 (60-90) additional family members per patient (120-150 total participants).

Specific Aim 1: Given that this is a Proof of Concept study, the primary aim of the project is to examine over a 4-week period (1) the feasibility of introducing the tablet and software to the patient and caregiver, (2) patients' and caregivers' willingness to use the tablet and software in their home, (3) the amount of time spent using the tablet and software, and (4) patients' and caregivers' satisfaction with the use of the tablet and software.

Hypothesis: It is anticipated that the tablet and software will be easily introduced and understood by the caregiver, that the patient will make consistent use of the tablet and device, and that both the patient and caregiver will be satisfied with the tablet and software.

Specific Aim 2: In addition to examining the usability and degree of interaction with the tablet and software, a secondary aim is to collect pilot data on the potential impact of using the tablet and software on patients' mood (anxiety, depression, and apathy), quality of life, and cognition, as well as the impact of the system on caregivers' mood, quality of life, and caregiver burden.

Hypothesis: It is anticipated that patients will improve in terms of their cognition, mood, and quality of life after the 4-week period. In particular, based on past studies of RT in patients with dementia, it is expected that the greatest effect on patients' report of depression symptoms will be found. In addition, it is also anticipated that caregiver burden will be lessened after the 4-week period.

Estimates have indicated that there are 5.2 million Americans suffering from Alzheimer's disease (AD), there are 1.4 million people in nursing homes and another 700,000 in residential care communities with about 50% of these individuals suffering from some form of dementia, and that the costs to the United States in caring for patients with AD or other dementias will be approximately $236 billion in 2016. The number of individuals with AD is expected to triple by the year 2050, which will result in approximately 4.2 million people with this disease, and the cost of patient care will also likely triple to $708 billion annually. The cognitive deficits and behavioral symptoms (e.g., depression, anxiety, and apathy) are difficult to treat in AD and other forms of dementia, and currently the first-line of treatment are pharmaceuticals, but these have met with only limited success.

Reminiscence Therapy (RT), Cognitive-Reminiscence Therapy (CRT), and Life Review Therapy (LRT) are behavioral interventions that involve the introduction of familiar pictures, music, or other materials to help individuals reminisce about their past experiences. These therapies have been shown to have a positive impact on mood and cognition in such populations as individuals with AD or other dementias, older adults with depression, and older adults with anxiety (Asiret & Kapucu, 2016; Gonzalez et al., 2015; Hseih et al., 2010; Hsu & Wang, 2009). A recent meta-analysis of 12 randomly controlled studies in older adults or patients with various forms of dementia demonstrated that RT significantly improved cognition and reduced depression after a brief trial (Huang et al., 2015). A major limitation of these therapies, however, is that it is typically provided in a formal therapy session, is only provided once a week, and is only provided within a limited time-frame, which greatly limits the consistent use of RT, CRT and LRT. Furthermore, these therapies often requires an individual to work one-on-one with a patient, which can be very time consuming for the caregiver and is often not practical in most settings. In addition, although the meta-analytic study described above found positive immediate effects of RT, the improvements in cognition and mood were limited and did not last after follow-up, suggesting that the effectiveness of such therapies will require consistent and ongoing implementation.

The application is an online-based story-sharing platform that allows users to record audio over photos as a way to share memories with family members who are suffering from a neurological or psychiatric condition. Similar to RT, CRT and LRT, the application is a potential therapeutic that allows patients to reminisce about their past, but does not have the structured time requirement or one-on-one administration that is needed with these formal therapies. Furthermore, the application is readily accessible and can easily be used on an ongoing basis by patients. The technology is unique in that it allows multiple family members, even if they are separated by time and place, to collaborate on the stories in just a few minutes a day. The platform transforms the short audio notes and individual photos into rich documentary-like stories that are then archived in a private and secured database. These stories can then be viewed very easily with a tablet whenever the patient chooses and the interface is very simple to operate. The application has the potential to be a practical and highly implementable adjunct behavioral intervention for a variety of patients, including those with dementia.

The proposed project will provide a Proof of Concept for the in-home use of the application in individuals with mild to moderate dementia.

Participants: The project will recruit 30 patients with mild to moderate dementia and their caregivers. Additionally, 2-3 family members will be asked who (1) own a smart phone or similar device and (2) are willing to participate, to upload photos and audio recordings throughout the 4-week period. This will result in approximately 120-150 participants. Participants will be recruited from Neuropsychological Associates, an outpatient neuropsychological clinic at UCSD that he directs. The inclusion criteria for patients are described in detail in Section 10 and the recruitment procedures are described in detail in Section 11. All persons involved in the study, whether it is the participant, caregiver, or family member, will be consented prior to uploading photos or audio recordings to the server where the files will be securely kept. Only the pictures and audio of those who consent to participate in the study will be uploaded. The study coordinator will be taking the necessary steps to ensure that random pictures of other family members who have not been consented will not be uploaded. More specifically, the study coordinator will have the opportunity to either discuss the confidential nature of the study at Visit 2 with the present family members, caregiver, and patient, or the study coordinator will discuss the same guidelines with the caregiver and patient while providing the names of the family members who have been consented over the phone. In addition, the study coordinator will monitor the audio, video, and photo uploads closely during Visit 2 while explaining to the participants that the software will be programmed to remind them to upload photos of only those individuals who have been consented throughout the 4-week period. Following this tutorial, the coordinator will then trust that family members will attend to the reminders that only consented individuals be included in the picture and that they will follow these guidelines for the remainder of the study.

Procedures: Patients and caregivers will be screened over the phone and if they are appropriate for the study and interested in participating they will be seen in their own homes by the study coordinator on three separate visits, which are detailed below. At the time of the phone screen, participants will be asked to think of 2-3 potential family members who may be willing to participate in the study so as to provide photos and audio that will serve as content for the stories. The names of these individuals will not be obtained during the phone screen, but rather, the caregiver will be asked to invite these family members to Visit 2, or if they are not local or willing/able to come to the patient's and caregiver's home on Visit 2, they will be provided with the study coordinator's phone number and asked to call the coordinator if they are interested in participating. Consent for these family members will be obtained either in person at the in-home Visit 2 or by phone (see Section 12 below) if they are unable/unwilling to attend. The specific procedures for each of the three in-home visits is described below:

In-Home Visit 1 (expected visit time is 1.5-2 hours): Patients and caregivers who are eligible for the study will be seen in their home, at which time the study coordinator will explain the details of the study, obtain informed consent or surrogate consent (if necessary) from both the patient and caregiver and administer a brief cognitive assessment to the patient and mood battery to both the patient and the caregiver (please see below for descriptions of each questionnaire). Family members who consent to be in the study will not be administered any tests or questionnaires. The battery of tests for the patient and caregiver will consist of the following measures:

Patient:
Feedback Form-Patient Version
Pocket vision screening and basic hearing screen.
Mattis Dementia Rating Scale (MDRS; Mattis, 1988)
Geriatric Depression Scale (GDS; Yesavage et al., 1982)
Geriatric Anxiety Inventory (GAI; Pachana et al., 2007)
Apathy Scale (AS; Starkstein et al., 1992)
Short Form Health Survey-36 (SF-36; Ware & Sherbourne, 1992)

Caregiver:
Feedback Form-Caregiver Version
Geriatric Depression Scale (GDS; Yesavage et al., 1982)
Geriatric Anxiety Inventory (GAI; Pachana et al., 2007)
Apathy Scale (AS; Starkstein et al., 1992)
Neuropsychiatric Inventory-Clinician (NPI-C; De Medeiros, K., et al., 2010)
Caregiver Burden Scale (CBS; Elmstahl et al., 1996)
Short Form Health Survey-36 (SF-36; Ware & Sherbourne, 1992)

These measures are well validated for their use in older individuals with and without dementia. Patients and caregivers are typically able to tolerate these measures with no difficulty. The detailed description of each measure is provided below.

Given the nature of the information gathered in regard to the patients' and caregivers' mood (depression, anxiety, and apathy), and given the vulnerability of the subjects (patients with dementia), a patient safety plan will be implemented to provide the proper follow up if the patient and/or caregiver were to report significant mood symptoms that constituted a risk of harming themselves or others.

During Visit 1, if the patient and caregiver have not yet identified 2-3 family members who are willing and able to participate in the study (i.e., provide photos and audio) the study coordinator will remind them to do so. If the family members have been identified, the study coordinator will ask the caregiver to invite these potential participants to attend Visit 2, at which time they will be consented if they are willing to participate. Family members who are unable to attend Visit 2, but are still willing to participate, will be asked by the caregiver to contact the study coordinator so that they can be consented by phone and provided with instructions on how to upload the photos and audio. The study coordinator will also ask that the caregiver attempt to identify up to 50 pictures to be uploaded to the application at Visit 2 (potentially with the help of the study coordinator if need be), and also ask any family member that is able to attend Visit 2 and is consented to do the same.

In-Home Visit 2 (expected visit time is 1.0 hour): A second in-home visit from the study coordinator will take place soon after Visit 1 (ideally the very next day but no more than 1 week after Visit 1). At this time the study coordinator will provide the patient with their tablet and provide a tutorial for the caregiver on how to operate the software using their personal device/smartphone. The study coordinator will also ensure where the materials (pictures and narratives) will be uploaded to the application, determine that the software is working appropriately, and instruct the patient and caregiver on the use of the system. It is important to note that the patient's tablet must be different from the caregiver's because their software configuration will be tailored to the patient. Specifically, the patient's tablet will only have the capability of playing the content but no other programs or applications, whereas the caregiver's device will have the capacity to upload the pictures and audio to the platform and will not have the capacity to view the content. Given that the caregivers will be using their own personal device, they will have the capacity to run any other programs or applications they desire.

During this visit, the 2-3 family members who were identified by the caregiver as possible participants and are able to attend this in-home visit will be consented and explained all procedures for uploading material to the application. As noted above, if the family members are not present at Visit 2, but are interested in participating by providing photos and audio, the caregiver will be asked to provide the family members with the study coordinator's contact information, if not done so already, so that they can contact the study coordinator and be told in detail by phone about the study, consented if they wish to participate, and provided with instructions on how to upload the content. Whether the family members be instructed in person or over the phone, once consented they will receive a secure, unidentifiable username and password, which will grant them access to the appropriate version of the software needed to participate in the study.

In-Home Visit 3 (expected visit time is 1.5-2 hours): The third in-home visit will be conducted after 4-weeks, at which time the study coordinator will re-administer the battery of tests described above under Visit 1. In addition, patients and caregivers will be given a questionnaire about their use and satisfaction with the tablet and software and return the tablet along with the self-reports back to the study coordinator (see Feedback Forms).

Interim Contact: The study coordinator will also contact the caregiver and patient by phone once a week during the 4-week study period to query them about the use of the tablet and software and answer any questions they might have about the system or any other concerns about the study. Additionally, patients, caregivers, and family members will be encouraged to contact the study coordinator with any questions they may have at any point during the duration of the study.

Research Material/Audio and Video Material: The research material to be evaluated in this study will be the Feedback Form for patients and caregivers, the frequency of the use of the tablet and software (recorded by the tablet), the cognitive measure, the mood measures, and the quality of life measures. The pictures and audio will be stored on a server but will never be analyzed in any manner. All of the images and videos stored in the application are stored with long (64 character) randomized filenames on a remote Amazon s3 server with no public index, thus, there is a high degree of security in terms of any outsider attempting to access the information. The filenames are stored in database and the only individual with access to that database is the software engineer, and the only reason he will access this information is to conduct routine maintenance and to debug live issues.

The creators of the software will only have access to de-identified test data (i.e., the various questionnaires administered at Visits 1 and 3, which will be shared electronically through the use of a secure storing-device once the study concludes. Following the completion of the study, all images or audio will be removed from the server.

Tablet and Software: the application is an online-based story-sharing platform that allows users to record audio over photos as a way to privately share and preserve their family history and personal legacy. Individuals are able to upload audio and pictures using their cell phone or computer, and the platform transforms the short audio notes and individual photos into rich documentary-like stories that are then archived in a private and secured database. The tablet and software are extremely user friendly and it is not anticipated that patients with dementia will have any problem using the device, particularly if caregivers are willing to help the patient use the system. The device is considered to be a non-significant risk device because it is a standard tablet that can be purchased by any individual and is not an FDA-regulated system. Furthermore, under the Code of Federal Regulations (CFR) 21 812.2, the tablet and software are consistent with the non-significant risk device requirements in that the device is noninvasive, it is not a medically established diagnostic product or procedure, it is not being tested for the purpose of diagnosing, curing, mitigating, or treating a disease, and it does not present a potential for serious risk to the health, safety, or welfare of a subject.

Sample Size and Power: Based on previous studies of RT, the effect size for a reduction in depression scores on the Geriatric Depression Scale was estimated to be 0.68 (Cohen's d). Given this effect size, the sample size would need to be 28, which would achieve a power level of 0.80 assuming a one tailed t-test at p<0.05. Thus, the 30 patients proposed in this study should be sufficient to detect differences. It should also be pointed out again that this is a Proof of Concept study with the primary aim of determining the feasibility of using the application with patients with dementia and their caregivers, and an examination in the improvement in mood, cognition, and quality of life is only a secondary aim.

Questionnaires: Feedback Form: This is a 5-10 minute feedback questionnaire that was designed to investigate the feasibility, frequency, adherence and overall likeability of the application software. As previously mentioned, this is a Proof of Concept study and our primary focus is to gain insight on whether or not the participants and caregivers enjoyed using the tablet and software. A modified version of a form used in a similar study (Moore, et al., 2015) involving the use of tablets has been created for the purposes of this study.

Mattis Dementia Rating Scale (MDRS; Mattis, 1988): This is a 20-30 minute measure consisting of 5 subscales (Attention, Initiation/Perseveration, Construction, Conceptualization, and Memory) that total to a maximum score of 144. The Mattis Dementia Rating Scale has been used extensively in a variety of cognitive studies dealing with patients with dementia. Although this test will not be used to make the diagnosis of dementia, it will be used as a determination of overall level of cognitive functioning.

Geriatric Depression Scale (GDS; Yesavage et al., 1982): This is a measure that has been used extensively with older populations. This is a brief 5-10 minute, 30-item questionnaire where the participants are asked to respond by answering “yes” or “no” regarding how they have felt over the past week. It has been well validated in its use with patients with dementia.

Geriatric Anxiety Inventory (GAI; Pachana et al., 2007): This measure is a 20-item, 5-10 minute, clinician-administered questionnaire involving anxiety symptom severity. Participants are asked to indicate whether they agree or disagree with certain anxiety related thoughts. All “agreed” responses are tallied where higher scores are indicative of higher anxiety levels. This scale has been used successfully in studies with geriatric populations with and without dementia.

Apathy Scale (AS; Starkstein et al., 1992): This scale consists of 14 items that are answered on a 4-point likert scale that asks questions about an individual's motivation, interest, and effort in their daily lives. It takes approximately 5-10 minutes to fill out. The scale was originally developed for use with patients with Parkinson's disease, but has been used successfully with patients with AD and stroke.

Short Form Health Survey-36 (SF-36; Ware & Sherbourne, 1992): The SF-36 is a 5-10 minute, clinician-administered questionnaire that assesses functional health and well-being scores across 8 subscales (physical functioning, role limitations due to physical functioning, bodily pain, general health, vitality, social functioning, role limitations due to emotional functioning, and mental health). This generic, health related, quality of life measure is often used when both disease and neurologically healthy populations are involved and has been widely used across the elderly population.

Neuropsychiatric Inventory-Clinician Rating Scale (NPI-C; De Medeiros, K., et al., 2010): The NPI-C is a 14-item, 10-15 minute, clinician-administered questionnaire that is administered separately to both the patient's caregiver (frequency, severity, and distress) and the patient (frequency) that assesses various neuropsychiatric symptoms (e.g., depression, anxiety, apathy, and sleep disturbance). In addition, the form allots for a clinician rating on a severity scale based on all available clinical and interview information that may have been observed. Psychiatric symptoms are identified using structured screening questions and positive responses are probed with structured follow-up questions. Follow-up questions are rated in terms of frequency on a scale of 1 to 4, severity on a scale of 1 to 3, and caregiver distress on scale of 1 to 5. It may not always be possible to interview the patient, and/or the patient may not be able to provide appropriate responses to all questions. In this instance, the total scores will be derived from the caregiver and clinician's responses. Each response pertaining to the individual scales for the caregiver, patient, and clinician, will be summed for a total score. The higher the scores within the reported neuropsychiatric domains, the greater the severity, frequency, and caregiver distress. The NPI-C has been used successfully in previous work to characterize psychiatric symptoms in patients with dementia.

Caregiver Burden Scale (CBS; Elmstahl et al., 1996): The CBS is 5-10 minute, 22-item questionnaire designed to reflect upon how people may or may not feel when taking care of another person.

The sample for the project will consist of 30 patients with a diagnosis of dementia, their caregivers, and 2-3 family members who are willing to provide content to the stories. This will result in 120-150 participants. The etiology of the dementia for the patients will likely be due to such conditions as Alzheimer's disease, Multi-Infarct Dementia, Parkinson's disease, or mixed etiologies. The inclusion and exclusion criteria for patients will be based on previous neuropsychological evaluation of the patient.

The inclusion criteria for the patients are the following:
Sixty-years or older.
Dementia (Major Neurocognitive Disorder) as diagnosed by the DSM-V.
Mild to Moderate cognitive deficits based on an MDRS total score of no less than 110.
Adequate hearing and vision to see tablet.
Caregiver who is available and willing to participate.
Patient and caregiver will not be traveling during the 4-week period of their participation.
Adequate comprehension and speaking of English (given test materials are in English only).

The exclusion criteria for the patients are the following:
Diagnosis of normal cognition, Mild Cognitive Impairment (MCI), or severe dementia (MDRS<110).
Current psychosis.
Current substance abuse.
Past or active symptoms of PTSD, Bipolar Disorder, or Schizophrenia.

Ideally caregivers will be the spouses or immediate relative of the patients, but when a spouse or relative is not available, only patients and their caregivers will be enrolled if the caregiver reports frequent interaction and daily communication with the patient. This will allow them to comfortably and accurately respond to the NPI regarding the patient's mood and behaviors and the ability to help the patient with the software and tablet when necessary. The inclusion and exclusion criteria for caregivers will be based on initial phone screen.

The inclusion criteria for the caregivers are the following:
Familiarity with the patient so as to answer questions about the patient's psychiatric functioning.
Daily contact with the patient to evaluate the patient's use of the tablet and device.
Ability to operate a simple computer tablet and software.
Willingness to help patient use system.
Adequate comprehension and speaking of English.

The exclusion criteria for the caregivers are the following:
History, based on self-report, of medical or cognitive problems that would prevent them from being able to help the patient with the tablet and software.
Current psychosis.
Current depression or anxiety.
Current substance abuse.

The inclusion criteria for the family members are the following:
Ownership of a smart phone or device
Ability to operate a simple computer tablet and software
Willingness to participate by uploading photos and audio recordings (i.e., 4-5 photos a week)
Adequate comprehension and speaking of English
Above the age of 18

The exclusion criteria for the family members are the following:
Current psychosis.
Current depression or anxiety.
Current substance abuse.

Patients, caregivers, and family members will not be excluded based on gender, race or ethnic background. Gender and ethnic composition of the samples will reflect that of the population of eligible patients that have been seen in an outpatient neuropsychological clinic. Women who are pregnant or are childbearing age will not be included because of the focus on patients with dementia who are 65 years or older. Minors will not be included because of the age of the targeted patients, caregivers, and family members.

Sources of Materials: Data to be collected in the proposed project consist of scores on the various cognitive tasks and evaluation scores of the mood-based assessments. For each set of data, subjects will be given an identification number and the corresponding subject name for these identification numbers will be kept on a secured computer system.

There will not be any direct benefit to our participants for completing the questionnaires and participating in the interview; however, the goal is that they enjoy using the tablet and software and that they show improvements in their mood, quality of life, and cognition after the 4-week period.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. A method for optimizing a digital therapeutic for a user, the method comprising:
(a) providing the user with the digital therapeutic, wherein the digital therapeutic comprises a digital content comprising one or more aspects thematically related to the user, wherein the providing of the digital therapeutic is via a digital therapeutic device;
(b) receiving feedback from the user through the digital therapeutic device relating to the digital therapeutic;
(c) processing the received feedback to calculate a therapeutic effect for each aspect of the one or more aspects, wherein the therapeutic effect corresponds to behavioral and cognitive changes in the user; and
(d) based on the calculated therapeutic effect, adjusting the digital content to present more or less of an aspect of the one or more aspects to the user.

2. The method of claim 1, wherein the behavioral and cognitive changes correspond to an improvement in cognition and mood of the user, such that the adjusting of the digital content optimizes the digital therapeutic by presenting more of an aspect of the one or more aspects to the user.

3. The method of claim 1, wherein the adjusting of the digital content is performed automatically by the digital therapeutic device.

4. The method of claim 1, wherein the adjusting of the digital content comprises prompting a caregiver to provide additional content for the digital content based on the calculated therapeutic effect.

5. The method of claim 1, wherein the one or more aspects of the digital content correspond to a family member, friend, childhood home, love interest, schooling, summer camp, or vacation of the user.

6. The method of claim 1, wherein the digital content comprises a photograph, a sound recording, a video, or any combination thereof.

7. The method of claim 1, wherein the feedback comprises the user inputting a request to play, repeat, skip, or approve an aspect of the one or more aspects of the digital content.

8. The method of claim 1, wherein the feedback comprises a user facial expression or user eye movement.

9. The method of claim 8, wherein the facial expression corresponds to surprise, happiness, fear, apathy, anger, sadness, disgust, or contempt in the user.

10. The method of claim 1, wherein the receiving of the feedback comprises tracking a user interaction and/or attention to the digital content.

11. The method of claim 10, wherein the tracking of user attention comprises monitoring the digital therapeutic device angle, a hand steadiness by the user, a sudden movement by the user, interaction with the digital therapeutic device by the user, eye gaze, eye movement, or any combination thereof.

12. A digital therapeutic device comprising:
(a) a user interface to i) provide a digital therapeutic to a user, wherein the digital therapeutic comprises a digital content comprising one or more aspects thematically related to the user, and ii) receive feedback from the user; and
(b) a processor operably coupled to the user interface, the processor configured to i) process the received feedback to calculate a therapeutic effect for each aspect of the one or more aspects, wherein the therapeutic effect corresponds to behavioral and cognitive changes in the user; and ii) adjust the digital content to present more or less of an aspect of the one or more aspects to the user.

13. The device of claim 12, wherein the user interface comprises i) a display to display the digital content, and/or ii) a speaker to transmit a sound recording to the user.

14. The device of claim 12, wherein the user interface comprises an image capture device to capture a facial expression or track eye movement of the user while the digital therapeutic is being provided.

15. The device of claim 14, wherein the facial expression corresponds to surprise, happiness, fear, apathy, anger, sadness, disgust, or contempt in the user.

16. The device of claim 14, wherein the image capture device is configured to track a user interaction and/or attention to the digital content.

17. The device of claim 16, wherein the tracking of user attention to digital content is based on the digital therapeutic device angle, a hand steadiness by the user, a sudden movement by the user, interaction with the digital therapeutic device by the user, eye gaze, eye movement, or any combination thereof.

18. The device of claim 12, further comprising a transceiver to transmit and receive information.

19. The device of claim 12, wherein the one or more aspects of the digital content correspond to a family member, friend, childhood home, love interest, schooling, summer camp, or vacation of the user.

20. A digital therapeutic device comprising:
(a) a user interface configured to display information and receive user input;
(b) a microphone configured to detect sound;
(c) a speaker configured to transmit sound;
(d) a video camera configured to record a patient's reaction to digital therapeutic content presented on the user interface;
(e) a transceiver configured to communicate with a database and a first user device; and
(f) a processor operatively coupled to the user interface, the microphone, the speaker, and the transceiver, wherein the processor is configured to:
(i) receive a first image from the database;
(ii) receive from the first user device a first message, wherein the first message includes a request for information related to the first image;
(iii) record via the microphone an audio recording that includes information related to the first image;
(iv) transmit the audio recording to the database;
(v) transmit to the database a request for the first image;
(vi) receive the first image with an identifier of the audio recording; and
(vii) cause the user interface to display the first image and simultaneously cause the speaker to play the audio recording.
